(19) United States
(12) Patent Application Publication
(10) Pub. No.: US 2018/0140482 A1
(43) Pub. Date: May 24, 2018

(54) Individual Packaging for and Absorbent Article

(71) Applicant: Productos Familia S.A., Medellin (CO)

(72) Inventors: Jaime Alberto Castaño Agudelo, Medellín (CO); Jerson Alexis Monsalve Villa, Medellín (CO); Juan Sebastián Sánchez Valdés, Medellín (CO)

(21) Appl. No.: 15/727,613

(22) Filed: Oct. 8, 2017

(30) Foreign Application Priority Data
Nov. 23, 2016 (CO) NC2016/0004462

(51) Int. Cl. A61F 13/551; A61F 13/15; A61F 13/475; A61F 13/537; B32B 27/32; B32B 27/36; B32B 27/30

(52) U.S. Cl. CPC A61F 13/5514; A61F 13/15747; A61F 13/15739; A61F 13/4755; A61F 2013/00702; B32B 27/32; B32B 27/36; B32B 27/306; A61F 2013/53786; A61F 13/53747

(57) ABSTRACT

The present invention relates to a package for an absorbent article with a permeable topsheet and an impermeable backsheet. The package comprises a flexible gas impermeable sheet on which the absorbent article is arranged such that the impermeable backsheet of the absorbent article is arranged on the flexible sheet. The flexible sheet is bent by describing a first section, a second section and a third section, the first section being folded over the second section such that upon folding of the absorbent article, the topsheet is arranged on itself, and the third section is folded over the first section. The longitudinal edges of the folded flexible sheet are sealed continuously by heat sealing, and the transverse edge is sealed continuously by adhesive seal.

### FIELD OF INVENTION

The present invention relates to the packagings for absorbent articles such as feminine protectors, sanitary napkins, diapers, among others. Specifically, the invention relates to a package for an absorbent article comprising volatile compounds or substances which can be modified given the exposure to the environment.

### DESCRIPTION OF THE STATE OF THE ART

Commonly, absorbent articles, such as female protectors, sanitary napkins, cloths, damp cloths and the like, are packaged individually to protect them from moisture or other environmental conditions.

In the state of the art, U.S. Pat. No. 6,911,022B2 and U.S. Pat. No. 6,375,645B1 are found, which disclose absorbent articles individually packaged.

U.S. Pat. No. 6,911,022B2 discloses an absorbent article, preferably a sanitary napkin with wings that are folded over the top sheet of the sanitary napkin.

The absorbent article is disposed in a sheath, such that it is affixed to the bottom surface of the absorbent article. The sheath's sheet and the sanitary napkin are folded in three parts with respect to two axes, such that one end overlaps on the opposite end. The edges of the sheath's sheet can be sealed by thermal and/or pressure and adhesive sealing. In one embodiment the adhesive layer is continuous, intermittent or by spot. The adhesive must have sufficient “peel” force to maintain the folding arrangement. Additionally, it is indicated that the material of the sheath's sheet is preferably liquid impermeable. In addition, some materials such as polymer films, film laminates, paper, nonwoven, among others are referred to.

U.S. Pat. No. 6,375,645B1 discloses an individually packaged absorbent article having a pair of first fins and a pair of second fins with fasteners such as adhesive fasteners. The fins are attached to the main body of the absorbent article along the longitudinal direction and extend laterally from the longitudinal edges of the absorbent article. It also discloses a sheath comprising a main sheath's sheet and covers for the fin fasteners. The main sheath's sheet has a pair of longitudinal edges, a pair of end edges, an inner surface facing the main body of the absorbent article and an outer surface. The main sheath's sheet and the absorbent article comprise two transverse axes and three regions divided by the two axes. The sheet and the absorbent article are folded around the axes, such that one of the end regions is bent over the region of the opposite end. After the folds are made, the longitudinal edges of the main sheath's sheet are sealed by any known technique such as thermal sealing, adhesives or ultrasound. A fastener or a fin is provided at the visible outer edge of the main sheath's sheet to facilitate the opening of the sheath. The absorbent article may be affixed to the main sheath's sheet by adhesives. The main sheath's sheet is liquid impermeable.

It is notable from prior art that there are no packagings for absorbent articles comprising volatile compounds or substances which can be modified in the event of exposure to the environment. The foregoing given the disclosed packages do not comprise continuous seals and/or liquid impermeable packaging materials, thereby allowing the escape of gases and exposure to the environment. In addition, in the case of liquid impermeable packaging materials, the volatile substances may escape the package or the gases may permeate the packaging, allowing contact with the absorbent article contained therein.

### BRIEF DESCRIPTION OF THE INVENTION

The present invention relates to a package for an absorbent article, including an absorbent article with a permeable topsheet and an impermeable backsheet, the package comprises:
a gas impermeable flexible sheet, the absorbent article is disposed in the flexible sheet such that the impermeable backsheet of the absorbent article is disposed on the flexible sheet of the packaging;
wherein the flexible sheet is folded by describing a first section, a second section and a third section, the first section is folded over the second section such that upon folding the absorbent article, the permeable topsheet of the absorbent article is disposed thereon and the third section is folded over the first section;
wherein the longitudinal edges of the folded flexible sheet are sealed continuously by heat sealing, and the transverse edge is continuously sealed by adhesive seal.

In one embodiment of the invention, the absorbent article has a siliconized sheet, and the flexible sheet of the package has at least one anchoring means. The siliconized sheet is adhered to the impermeable backsheet of the absorbent article, and the anchoring means is disposed in one of the three sections of the flexible sheet of the package. By arranging the absorbent article in the flexible sheet, the siliconized sheet of the absorbent article is adhered to the anchoring means and therewith the absorbent article is affixed to the flexible sheet.

The process for forming the package from the flexible sheet and containing the absorbent article comprises the following steps:
a) arranging the absorbent article in the flexible sheet, the backsheet of the absorbent article is arranged on the flexible sheet;
b) folding the flexible sheet with the absorbent article into three sections, a first section is folded over a second section such that upon folding the absorbent article, the topsheet is arranged on itself, and the third section is folded over the first section;
c) heat sealing the longitudinal edges of the folded flexible sheet; and
d) adhesive sealing the transverse edge of the folded flexible sheet.

### DESCRIPTION OF THE FIGURES

FIG. 1 shows an isometric view of an embodiment of the invention in which the absorbent article is disposed on the sheet, and the three sections are displayed.

FIG. 2 shows an isometric view of an embodiment of the invention in which the anchoring means and the siliconized sheet are displayed.

### DETAILED DESCRIPTION OF THE INVENTION

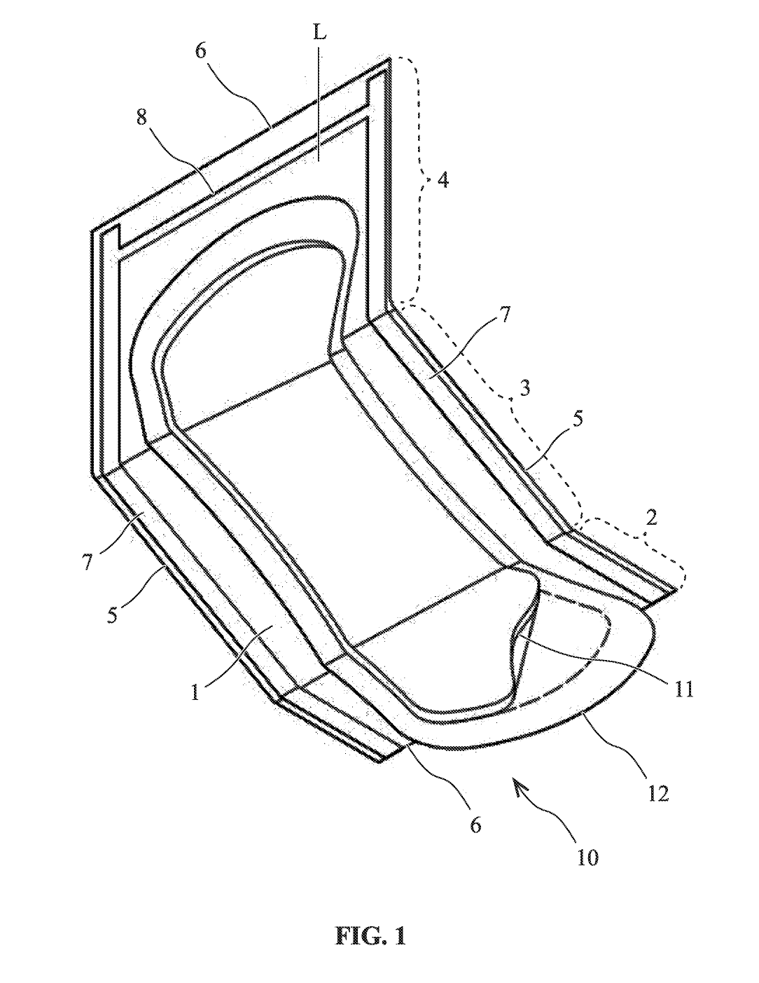

Referring to FIG. 1, the package comprises a flexible sheet (1) which is gas impermeable. The absorbent article (10) is arranged on the flexible sheet (1) in such a way that the impermeable backsheet (12) of the absorbent article (10) is arranged on the flexible sheet (1). To configure the package, the flexible sheet (1) is folded by describing a first section (2), a second section (3) and a third section (4). The first section (2) is folded over the second section (3) in such a way that upon folding of the absorbent article, the topsheet (11) thereof is arranged on itself. And the third section (4) is folded over the first section (2). Such as the folded flexible sheet (1) describes two longitudinal edges (5) and two transverse edges (6), a first transverse edge (6) is overlapped over the second transverse edge (6).

The flexible sheet (1) is formed between two and three layers joined together. In the two-layer mode, the flexible sheet (1) is formed of a layer of metallized polypropylene or bi-oriented metallized polypropylene, and a layer of bioriented polypropylene or polypropylene. In the three layer mode, two layers of polyethylene are arranged, and among them a layer of ethylene-vinyl alcohol or polyester.

Referring to FIG. 1, the edges of the flexible sheet (1) folded with the absorbent article (10) therein are continuously sealed. The longitudinal edges (5) are sealed by a heat seal (7), one on each longitudinal edge (5). The transverse edges (6) are sealed by adhesive (8). The longitudinal edges of the first section (2), the second section (3) and the third section (4) are joined by the heat seal (7) of the longitudinal edges (5). The adhesive (8) is arranged on the first transversal edges (6) disposed in the third section (4) and in this way close the package but allowing its easy opening.

For understanding of the present invention, the term continuously sealed is referred to uninterrupted sealed which does not have cessation.

The location of the heat seal (7) is such that it is not located on the absorbent article (10), and is preferably located in the vicinity of the periphery of the longitudinal edges (5). The heat seal (7) has a width greater than or equal to 4 mm, therefore an outer edge and an inner edge are observed for each heat seal (7).

The adhesive has a width greater than or equal to 2 mm, therefore an outer edge and a lower edge are observed. The outer edge is located at a distance L between 5 to 8 mm, such that a non-adhesive surface is provided which serves as a support for the grip at the time of accessing the absorbent article contained in the package.

In one embodiment of the invention, with reference to FIG. 1, the adhesive extends between the inner edges of the heat seals (7).

In one embodiment of the invention (not shown), the adhesive extends between the outer edges of the heat seals (7).

For understanding of the present invention, the term anchoring means (9) is related to means to keeping the absorbent article (10) fix to the flexible sheet (1).

In one embodiment of the invention, the absorbent article (10) has a siliconized sheet (13), and the flexible sheet (1) has at least one anchoring means (9). The siliconized sheet (13) adheres to the impermeable backsheet (12) of absorbent article (10), and the anchoring means (9) is arranged in one of the three sections of the flexible sheet (1). As the absorbent article (10) is arranged on the flexible sheet (1), the siliconized sheet (13) is adhered to the anchoring means (9) and thereby the absorbent article is attached to the flexible sheet (1). The anchoring means (9) is selected from the group consisting of adhesive, velcro and a combination thereof.

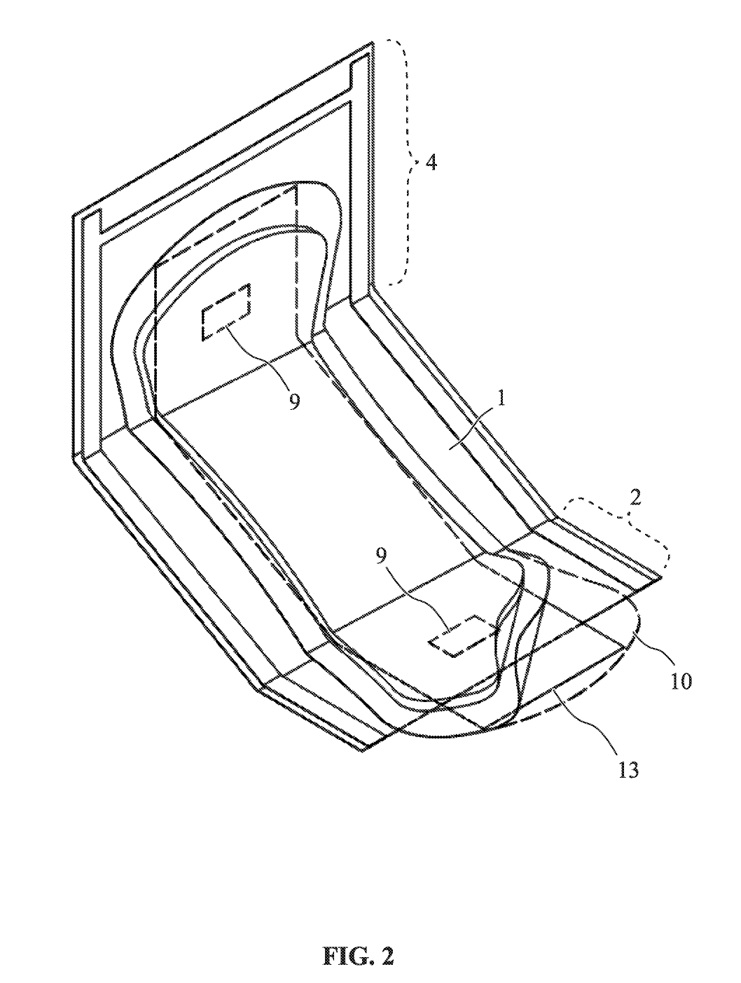

Referring to FIG. 2, the absorbent article (10) has a siliconized sheet (13), and the flexible sheet (1) has two anchoring means (9). The siliconized sheet (13) adheres to the impermeable backsheet (12) of the absorbent article (10). The two anchoring means (9) are arranged in the flexible sheet (1), one in the first section (2) and another in the third section (4).

In one embodiment (not shown), the flexible sheet (1) has three anchoring means (9), one per each section.

In one embodiment of the invention (not shown), the siliconized sheet (13) is attached to the flexible sheet (1). In this embodiment, the backsheet (12) is arranged on the siliconized sheet (13).

The process for forming the package from the flexible sheet (1) and containing the absorbent article (10) comprises the following steps:
a) Arranging the absorbent article (10) on the flexible sheet (1), the backsheet (12) is arranged on the flexible sheet (1);
b) Folding the flexible sheet (1) with the absorbent article (10) into three sections, a first section (2) is folded over a second section (3) in such a way that upon folding of the absorbent article (10), the topsheet (11) is arranged on itself, and the third section (4) is folded over the first section (2);
c) heat sealing the longitudinal edges (5) of the folded flexible sheet (1); and
d) adhesive sealing the transverse edge (6) of the folded flexible sheet (1).

Preferably, after folding the flexible sheet (1) in step (b), the folding flexible sheet (1) and the absorbent article (10) are subject to pressure in order to extracted the air comprised between the flexible sheet (1) and the absorbent article (10).

In step (c), the heat sealing is performed at a temperature between 124° C. and 147° C., and a pressure between 313.6 kPa and 509.6 kPa.

In step (d), the adhesive has a bond strength greater than 392N*m. One adhesive that complies with this condition is the adhesive marketed by Fuller under reference HL2110X.

### Example 1

A package for a protector type absorbent article (10) was configured. The protector had a volatile menthol lotion that generates a freshness effect on the wearer. The dimensions of the protector were:
length: 150 mm
width: 65 mm
thickness: 2.5 mm

A siliconized sheet was adhered to the permeable backsheet (12) of the absorbent article (13).

The flexible sheet (1) was formed by a layer of bi-oriented metallized polypropylene and a bioriented polypropylene layer. The dimensions of the flexible sheet (1) were 150 mm long, 85 mm wide and a thickness of 38 μm. The flexible sheet (1) had a grammage of 33 g/m².

The protector was arranged on the flexible sheet (1) centered with respect to the longitudinal centers of both elements. The flexible sheet (1) was folded into three sections, which had the following lengths:
first section (2): 30 mm
second section (3): 60 mm
third section (4): 60 mm

The protector was arranged on the flexible sheet (1) protruding from these 17 mm, measured from the first section (2).

The protector was attached to the flexible sheet (1) by two anchoring means (9), one in the first section (2) and the other in the third section (4). Each anchoring means (9) was 13 mm wide and 7 mm long. The anchoring means (9) was configured with Fuller adhesive under reference 5600L. The anchoring means (9) located in the first section (2) was arranged to 39.5 mm from the edge of the first section (2). The anchoring means (9) located in the third section (4) was arranged to 10 mm from the edge of the third section (4).

Each heat seal (7) had a width of 5 mm. The outer edge of each heat seal (7) was located to 2 mm from the longitudinal edges (5).

The adhesive (8) had a width of 3 mm and was arranged at a distance L equal to 6 mm. The adhesive used was Fuller under reference HL2110X.

### Example 2

The process for forming the package of example 1 comprised the following steps:
a) The protector was arranged on the flexible sheet (1), the backsheet (12) was arranged on the flexible sheet (1);
b) The flexible sheet (1) was folded with the protector into three sections so that the first section (2) was folded over the second section (3) in such a way that when folding the protector, the topsheet (11) is arranged on itself, and the third section (4) was bent over the first section (2);
c) The longitudinal edges (5) of the folded flexible sheet (1) were heat sealed; and,
d) The transverse edge (6) of the folded flexible sheet (1) was adhesive sealed.

After performing the folds of step (b), the air comprised between flexible sheet (1) and protector was extracted, as the assembly was pressed by a neoprene strip.

This process was performed on a FAMECCANICA FNL-10 machine at a speed of 1200 units per minute. The heat sealing was performed at a temperature of 127° C. and a pressure of 460 kPa

### Example 3

A package for an absorbent article (10) sanitary napkin was configured. The sanitary napkin had a volatile menthol lotion that generates a freshness effect on the wearer. The dimensions of the sanitary napkin were:
length: 232 mm
width: 93 mm
thickness: 3.8 mm

The fins of the sanitary napkin were bent towards the permeable topsheet and were secured in this position to a siliconized sheet with adhesive fuller 5600L.

A siliconized sheet (13) was adhered to the permeable backsheet (12).

The flexible sheet (1) was formed by a layer of metallized BOPP and a layer of bioriented polypropylene. The dimensions of the flexible sheet (1) were 220 mm long, 116 mm wide and a thickness of 38 μm. The flexible sheet (1) had a grammage of 33 g/m².

The protector was arranged on the flexible sheet (1) centered with respect to the longitudinal centers of both elements. The flexible sheet (1) was folded into three sections, which had the following lengths:
first section (2): 47 mm
second section (3): 87 mm
third section (4): 86 mm

The protector was arranged on the flexible sheet (1) displaced 19 mm, measured from the transverse edge of the third section (4).

The protector was attached to the flexible sheet (1) by three anchoring means (9), one for each section. Each anchoring means (9) was 13 mm wide and 7 mm long. The anchoring means (9) was configured with Fuller adhesive under reference 5600L. The anchoring means (9) located in the first section (2) was arranged to 39.5 mm from the edge of the first section (2). The anchoring means (9) located in the third section (4) was arranged to 10 mm from the edge of the third section (4). The anchoring means (9) located in the second section (3) was arranged in the geometric center of this.

Each heat seal (7) had a width of 5 mm. The outer edge of each heat seal (7) was located at 2 mm from the longitudinal edges (5).

The adhesive (8) had a width of 3 mm and was arranged at a distance L equal to 6 mm. The adhesive used was Fuller HL2110X.

### Example 4

The process for forming the packaging of example 3 comprised the following steps:
a) arranging the sanitary napkin on the flexible sheet (1), and arranging the backsheet (12) on the flexible sheet (1);
b) folding the flexible sheet (1) with the sanitary napkin into three sections so that the first section (2) is folded over the second section (3) in such a way that when the sanitary napkin is folded, the topsheet (11) is arranged on itself, and the third section (4) is folded over the first section (2);
c) heat sealing the longitudinal edges (5) of the folded flexible sheet (1); and,
d) adhesive sealing the transverse edge (6) of the folded flexible sheet (1).

Prior to performing step (a), the wings have been folded and fixed to the siliconized sheet.

After performing the folds of step (b), the air comprised between the flexible sheet (1) and the sanitary napkin was extracted, as the assembly was pressed by a neoprene strip.

This process was performed on a FAMECCANICA FNL-10 machine at a speed of 700 units per minute. The heat sealing was performed at a temperature of 147° C. and a pressure of 380 kPa.

It should be understood that the present invention is not limited to the embodiments described and illustrated. As will be apparent to a person skilled in the art, there are possible variations and modifications which do not depart from the spirit of the invention, which is only found defined by the following claims.

1. A package for an absorbent article with a permeable topsheet and an impermeable backsheet, comprising:
a gas impermeable flexible sheet, the absorbent article is arranged on the flexible sheet such that the impermeable backsheet is arranged on the flexible sheet;
wherein the flexible sheet is folded by describing a first section, a second section and a third section, the first section is folded over the second section such that upon folding of the absorbent article, the topsheet is arranged on itself, and the third section is folded over the first section such as the folded flexible sheet describes two longitudinal edges and two transverse edge, a first transverse edge is overlapped over the second transverse edge;
wherein the longitudinal edges of the folded flexible sheet are sealed continuously by heat seal, and the first transverse edge is sealed continuously to the first section by adhesive seal to the first section.

2. The package of claim 1, wherein the flexible sheet is formed of:
a first layer of metallized polypropylene or metallized bioriented polypropylene; and,
a second layer of bioriented polypropylene or polypropylene.

3. The package of claim 1, wherein the flexible sheet is formed of:
a first layer of polyethylene;
a second layer of ethylene-vinyl alcohol or polyester, and,
a third layer of polyethylene.

4. The package of claim 1, comprising:
a siliconized sheet adhered to the backsheet; and,
an anchoring means by which the siliconized sheet is affixed to the flexible sheet.

5. The package of claim 4, wherein the anchoring means is selected from the group consisting of adhesive, velcro and a combination thereof.

6. The package of claim 4, comprising an anchoring means in the first section and an anchoring means in the third section.

7. The package of claim 4, comprising an anchoring means in the first section, an anchoring means in the second section and an anchoring means in the third section.

8. The package of claim 1, wherein the flexible sheet has a siliconized layer and the backsheet is arranged on the siliconized layer.

9. The package of claim 1, wherein the adhesive seal extends between the inner edges of the heat seal.

10. The package of claim 1, wherein the adhesive seal extends between the outer edges of the heat seal.

11. The package of claim 1, wherein the adhesive seal is arranged at an L distance from the outer edge of the third section.

12. The package of claim 1, wherein the thickness of the adhesive seal is greater than 2 mm.

13. The package of claim 1, wherein the thickness of the heat seal is greater than 4 mm.

14. A process for forming a flexible sheet packaging for an absorbent article having a permeable topsheet and an impermeable backsheet comprising the steps of:
a) arranging the absorbent article on the flexible sheet, the backsheet is arranged on the flexible sheet;
b) folding the flexible sheet with the absorbent article into three sections, a first section is folded over a second section such that upon folding of the absorbent article, the topsheet is arranged on itself, and the third section is folded over the first section such as the folded flexible sheet describes two longitudinal edges and two transverse edge, a first transverse edge is overlapped over the second transverse edge;
c) heat sealing the longitudinal edges of the folded flexible sheet; and,
d) adhesive sealing the longitudinal edge of the folded flexible sheet.

15. The process of claim 14, wherein the folded flexible sheet of step (b) and the absorbent article are subject to pressure.

16. The process of claim 14, wherein step (c) is performed at a temperature between 124° C. and 147° C., and a pressure between 313.6 kPa bar and 509.6 kPa.

17. The process of claim 14, wherein the adhesive of step (d) has a bond strength greater than 392N*m.
